(19) United States
(12) Patent Application Publication
(10) Pub. No.: US 2006/0020005 A1
(43) Pub. Date: Jan. 26, 2006

(54) Process for preparing losartan

(76) Inventors: Nadimpally Satyavarahala Raju, Hyderabad (IN); Koilkonda Purandhar, Hyderabad (IN); Suthrapu Sashikanth, Warangal (IN); Lekkala Reddy Amarnath, Hyderabad (IN); Vecha Sravanthi, Khammam (IN); Golla China Mala Kondaiah, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/186,506

(22) Filed: Jul. 21, 2005

Related U.S. Application Data
(60) Provisional application No. 60/590,067, filed on Jul. 21, 2004.

(51) Int. Cl. A61K 31/4178; C07D 403/02

(52) U.S. Cl. 514/381; 548/253

(57) ABSTRACT

A process for preparing losartan, comprising reacting trityl losartan with an aqueous acid.

### CROSS-REFERENCE TO RELATED APPLICATION

This application derives priority from copending U.S. Provisional Application No. 60/590,067 filed on Jul. 21, 2004, the entire content of which is incorporated herein by this reference.

### INTRODUCTION TO THE INVENTION

The present invention relates to an improved process for the preparation of losartan free acid, which is useful for making losartan potassium, chemically known as 2-butyl-4-chloro-1-[p-(o-1H-tetrazol-5-ylphenyl)benzyl]imidazole-5-methanol monopotassium (which is hereinafter referred to using the adopted name “losartan potassium”) and having the Formula I.

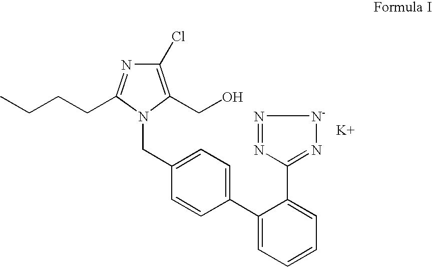

Losartan potassium is an angiotensin II receptor (type AT₁) antagonist and is commercially available in pharmaceutical products sold using the trademark COZAAR™.

U.S. Pat. No. 5,138,069 discloses that the losartan free acid preparation in the presence of methanol as a solvent contains 2-Butyl-4-chloro-1[[2′-(1H-tetrazole-5-yl)[1,1′-biphenyl]-4-yl]methyl]-1H-imidazole-5-methoxy methyl ether as an impurity, at a level of about 1.2% or more, and this impurity also carries into the final compound, e.g., losartan potassium.

Hence, there is a need to develop a simple, improved and cost effective process for preparing losartan which is commercially viable. Therefore the present invention provides a cost effective and commercially viable process, which involves treating trityl losartan with an acid in the presence of organic solvents.

### SUMMARY OF THE INVENTION

An aspect of present invention, therefore, is a process for making losartan free acid which is substantially free of 2-Butyl-4-chloro-1-[[2′-(1H-tetrazole-5-yl)[1,1′-biphenyl]-4-yl] methyl]-1H-imidazole-5-methoxy methyl ether, comprising reacting 2-butyl-4-chloro-5-hydroxymethyl-1-[(2-triphenylmethyl-2H-tetrazole-5-yl)biphenyl-4-yl)methyl] 1H-imidazole (trityl losartan) of Formula III 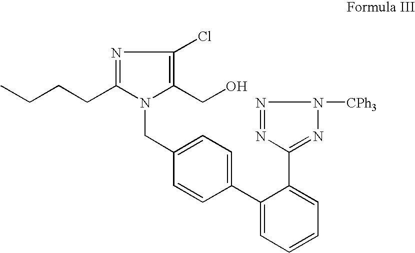
with an aqueous acid in the presence of organic solvents to give 2-butyl-4-chloro-1-[p-(o-1H-tetrazol-5-ylphenyl) benzyl] imidazole-5-methanol (losartan free acid) of Formula II,

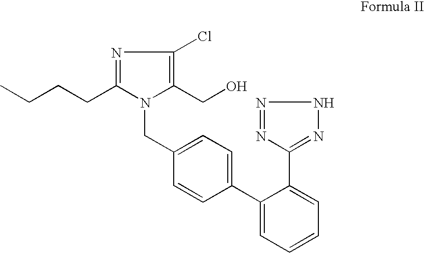

In another aspect of the present invention, therefore, there is provided a process for making losartan potassium of Formula I, which is substantially free of 2-Butyl-4-chloro-1[[2′-(1H-tetrazole-5-yl)[1,1′-biphenyl]-4-yl]methyl]-1H-imidazole-5-methoxy methyl ether, comprising reacting 2-butyl-4-chloro-1-[p-(o-1H-tetrazol-5-ylphenyl)benzyl]imidazole-5-methanol (ILosartan free acid) of Formula II, 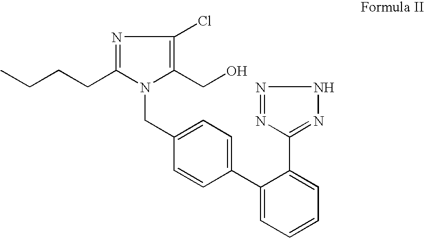
with an aqueous base in the presence of an organic solvent to give 2-butyl-4-chloro-1-[p-(o-1H-tetrazol-5-ylphenyl)benzyl]imidazole-5-methanol monopotassium (Losartan potassium) of Formula I,

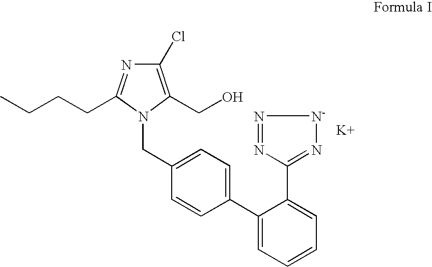

### DETAILED DESCRIPTION

In one aspect of the present invention, therefore, there is provided a process for making losartan free acid, an intermediate of losartan potassium, which is substantially free of 2-Butyl-4-chloro-1[[2′-(1H-tetrazole-5-yl)[1,1′-biphenyl]-4-yl]methyl]-1H-imidazole-5-methoxy methyl ether, comprising reacting 2-butyl-4-chloro-5-hydroxymethyl-1-[(2-triphenylmethyl-2H-tetrazole-5-yl)biphenyl-4-yl)methyl]1H-imidazole (trityl losartan) of Formula III 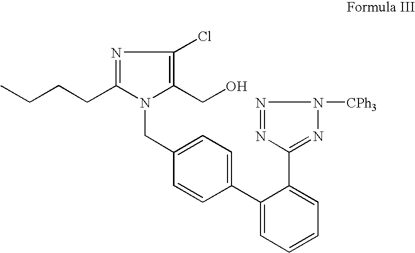
with an aqueous acid in the presence of an organic solvent to give 2-butyl-4-chloro-1-[p-(o-1H-tetrazol-5-ylphenyl)benzyl]imidazole-5-methanol (losartan free acid) of Formula II,

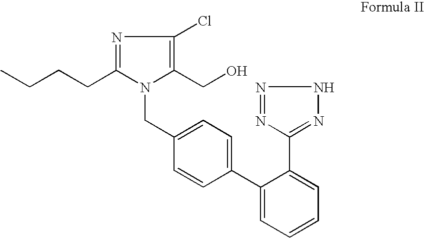

Suitable aqueous acids include but are not limited to methanesulfonic acid, trifluoroacetic acid, trifluoromethanesulfonic acid, sulfuric acid, phosphoric acid, benzenesulfonic acid, p-toluenesulfonic acid, p-chlorobenzene-sulfonic acid, hydrochloric acid, acetic acid, formic acid and the like.

Suitable organic solvents include, but are not limited to, members from the classes: ketonic solvents such as acetone, ethylmethyl ketone, methyl isobutyl ketone and the like; esters such as ethyl acetate, n-propyl acetate, n-butyl acetate, t-butyl acetate and the like; ether solvents such as diethyl ether, dimethylether, di-isopropylether, methyltertiarybutyl ether, tetrahydrofuran, 1,4-dioxane and the like; hydrocarbon solvents such as toluene, xylene and the like; nitrile solvents such as acetonitrile, propionitrile and the like; halogenated solvents such as dichloromethane,1,2-dichloroethane, chloroform, carbon tetrachloride and the like; aprotic polar solvents such as dimethylsulfoxide (DMSO), N,N-dimethylformamide (DMF), N,N-dimethylacetamide and the like; or mixtures of any two or more thereof in various proportions.

The reaction can be carried out at about 10 to 50° C. or 20 to 35° C.

Losartan free acid prepared according to this embodiment has a low level of the 2-Butyl-4-chloro-1[[2′-(1H-tetrazole-5-yl)[1,1′-biphenyl]-4-yl]methyl]-1H-imidazole-5-methoxy methyl ether impurity as determined by HPLC. It contains less than about 1.2 area-% or about 0.15 area-%, or about 0.05 area-%, or is substantially free of 2-n-Butyl-4-chloro-1[[2′-(1H-tetrazole-5-yl)[1,1′-biphenyl]-4-yl] methyl]-1H-imidazole-5-methoxy methyl ether.

Losartan free acid prepared according to this embodiment further can be used to prepare its salts such as losartan potassium of formula I with high purity. The potassium (or sodium, calcium, ammonium, etc. salts) can be prepared by reacting losartan free acid with the respective bases in presence of suitable organic solvents and conditions.

The process for making losartan potassium of Formula I, which is substantially free of 2-Butyl-4-chloro-1[[2′-(1H-tetrazole-5-yl)[1,1′-biphenyl]-4-yl] methyl]-1H-imidazole-5-methoxy methyl ether, comprises reacting 2-butyl-4-chloro-1-[p-(o-1H-tetrazol-5-ylphenyl)benzyl]imidazole-5-methanol (losartan free acid) of Formula II, 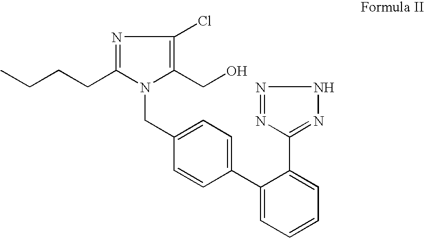
with an aqueous base in the presence of an organic solvent to give 2-butyl4-chloro-1-[p-(o-1H-tetrazol-5-ylphenyl)benzyl]imidazole-5-methanol monopotassium (losartan potassium) of Formula I,

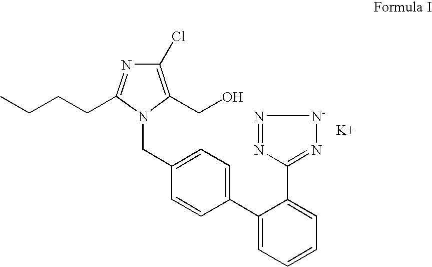

Suitable aqueous bases include but are not limited to potassium hydroxide, potassium carbonate, potassium secondary butoxide, potassium tertiary butoxide and the like.

Suitable organic solvents include, but are not limited to, members from the classes: alcohol solvents such as ethanol, isopropylalcohol, n-propanol, n-butanol, isobutyl alcohol, tertiary butyl alcohol and the like; ketonic solvents such as acetone, ethylmethyl ketone, methyl isobutyl ketone and the like; esters such as ethyl acetate, n-propyl acetate, n-butyl acetate, t-butyl acetate and the like; ether solvents such as diethyl ether, dimethylether, di-isopropylether, methyltertiarybutyl ether, tetrahydrofuran, 1,4-dioxane and the like; hydrocarbon solvents such as toluene, xylene and the like; nitrile solvents such as acetonitrile, propionitrile and the like; halogenated solvents such as dichloromethane,1,2-dichloroethane, chloroform, carbon tetrachloride and the like; aprotic polar solvents such as dimethylsulfoxide (DMSO), N,N-dimethylformamide (DMF), N,N-dimethylacetamide and the like; or mixtures of any two or more thereof in various proportions.

The reaction can be carried out at about 10 to 50° C. or 20 to 35° C.

Losartan potassium prepared according to this embodiment has a low level of the 2-Butyl-4-chloro-1[[2′-(1H-tetrazole-5-yl)[1,1′-biphenyl]-4-yl]methyl]-1H-imidazole-5-methoxy methyl ether impurity as determined by HPLC. It contains less than about 1.2 area-%, or less than about 0.15 area-%, or less than about 0.05 area-%, or is substantially free of, 2-n-Butyl-4-chloro-1[[2′-(1H-tetrazole-5-yl)[1,1′-biphenyl]-4-yl]methyl]-1H-imidazole-5-methoxy methyl ether.

The following examples are only illustrative of certain aspects of the invention and are not intended to limit the scope of the invention

### EXAMPLE-1

Preparation of 2-Butyl-4-Chloro-1-[P-(0-1H-Tetrazol-5-Ylphenyl) Benzyl]Imidazole-5-Methanol (Losartan Free Acid) Formula (II)

200 ml of Dichloromethane and 50 grams of 2-butyl-4-chloro-5-hydroxymethyl-1-[(2-triphenylmethyl-2H-tetrazole-5-yl) biphenyl-4-yl) methyl] 1 H-imidazole (trityl losartan) were charged in a clean and dry round bottom flask. A solution of 18 ml of water and 18 ml of 12N hydrochloric acid was added to the above reaction mass at about 25 to 35° C. followed by stirring for about 60 to 90 min. 18 ml of water was charged and the pH of the resultant reaction mass was adjusted to about 8 to 9 with 15 ml of caustic lye followed by separation of organic and aqueous layers. The aqueous layer was washed with 200 ml of dichloromethane and followed by addition of methanol to the aqueous layer. The pH of the aqueous layer was adjusted to about 5 to 5.5 with 6.1 ml of hydrochloric acid at about 40 to 45° C. The reaction mixture was stirred for about 45 to 60 min. at about 40 to 45° C. followed by filtration of the separated solid. The solid obtained was washed with 50 ml of water and dried at about 70 to 75° C. for about 3 to 4 hours to afford title compound which was substantially free of 2-Butyl-4-chloro-1-[[2′-(1H-tetrazole-5-yl)[1,1′-biphenyl]-4-yl]methyl]-1H-imidazole-5-methoxymethyl ether and had a purity 99.8% by HPLC.

### EXAMPLE-2

Preparation of 2-Butyl-4-Chloro-1-[P-(0-1H-Tetrazol-5-Ylphenyl) Benzyl]Imidazole-5-Methanol (Losartan Free Acid) Formula (II)

1500 ml of Acetonitrile and 500 grams of 2-butyl-4-chloro-5-hydroxymethyl-1-[(2-triphenylmethyl-2H-tetrazole-5-yl) biphenyl-4-yl) methyl] 1H-imidazole (trityl losartan) were charged to a clean and dry round bottom flask. A mixture of 120 ml 12 N hydrochloric acid and 260 of water were added to the above reaction mass for about 45 to 60 min. at about 20 to 25° C. The resultant reaction mass was stirred for about 4 to 6 hours followed by pH adjustment to about 8 to 8.5 at about 20 to 25° C. with 110 ml of caustic lye followed by addition of water (2500 ml) for about 30 to 45 minutes under stirring. Separated solid was filtered and the solid obtained was washed with 500 ml of water. The filtrate was washed with 2×1000 ml of dichloromethane followed by separation of the aqueous layer. Aqueous layer washed with 500 ml of dichloromethane followed by separation of organic and aqueous layers. Aqueous layer was heated to about 35-45° C. followed by adjustment of pH to about 5 to 5.5 with 90 ml of 12N hydrochloric acid followed by stirring for about 45 to 60 minutes at about 35 to 45° C.

Resultant suspension was cooled to about 25-35° C. followed by stirring for about 30-60 min. Separated solid was filtered and the solid obtained was washed with 500 ml of water. Wet solid and 1500 ml of water were charged in a clean and dry round bottom flask and was stirred for about 45-60 min. Solid was filtered and was washed with 500 ml of water and again the resultant wet solid and 1500 ml of water were charged and was stirred for about 30-60 min. Solid was filtered and was washed with 500 ml of water and then dried at about 65-75° C. for about 3 to 5 hours to afford 274.7 g of title compound which was substantially free of 2-Butyl4-chloro-1[[2′-(1H-tetrazole-5-yl)[1,1′-bipHenyl]-4-yl] methyl]-1H-imidazole-5-methoxy methyl ether and had a purity 99.6% by HPLC.

### EXAMPLE-3

Preparation of 2-Butyl-4-Chloro-1-[P-(0-1H-Tetrazol-5-Ylphenyl) Benzyl]Imidazole-5-Methanol Monopotassium Salt (Losartan Potassium) (Formula I)

25 g of 2-butyl-4-chloro-1-[p-(o-1H-tetrazol-5-ylphenyl)benzyl]imidazole-5-methanol (losartan free acid) and 75 ml of isopropyl alcohol were charged to a clean and dry round bottom flask and the mixture was stirred for about 15-30 minutes followed by pH adjustment to about 9 to 9.5 by the addition of a mixture of 3.98 g of potassium hydroxide, 2.5 ml of water and 25 ml of isopropyl alcohol under stirring over about 30-60 minutes. The mixture was stirred for about 1-2 hours, followed by charging 1 g of basic and active charcoal carbon and then stirring for about 30-45 minutes. The suspension was filtered on celite and the celite was washed with 25 ml of isopropyl alcohol followed by distillation of solvent at about 55-70° C. under vacuum over about 30-45 minutes. 125 ml of n-heptane was charged and the distillation was continued. 62.5 ml of n-heptane was charged and the resultant reaction mass was cooled to about 25-35° C. followed by stirring for about 30-60 minutes. Separated solid was filtered and the solid obtained was washed with 25 ml of n-heptane and then dried at about 90-95° C. for about 10-15 hours under vacuum, to afford 26 g of title compound with a purity by HPLC 99.7%.

We claim:

1. A process for preparing losartan free acid, comprising reacting trityl losartan with an aqueous acid.

2. The process of claim 1, wherein trityl losartan is present as a solution in an organic solvent.

3. The process of claim 1, wherein an aqueous acid comprises methanesulfonic acid, trifluoroacetic acid, trifluoromethanesulfonic acid, sulfuric acid, phosphoric acid, benzenesulfonic acid, p-toluenesulfonic acid, p-chlorobenzene-sulfonic acid, hydrochloric acid, acetic acid, or formic acid.

4. The process of claim 1, wherein trityl losartan is present as a solution in a ketone, an ester, an ether, a hydrocarbon, a halogenated solvent, an aprotic polar solvent, or a mixture of any two or more thereof.

5. Losartan free acid prepared by the process of claim 1.

6. Losartan free acid prepared by the process of claim 1 and having less than about 1.2 area-percent of 2-n-Butyl-4-chloro-1[[2′-(1H-tetrazole-5-yl)[1,1′-biphenyl]4-yl] methyl]-1H-imidazole-5-methoxy methyl ether, as measured by high performance liquid chromatography.

7. Losartan free acid prepared by the process of claim 1 and having less than about 0.15 area-percent of 2-n-Butyl-4-chloro-1[[2′-(1H-tetrazole-5-yl)[1,1′-biphenyl]-4-yl] methyl]-1H-imidazole-5-methoxy methyl ether, as measured by high performance liquid chromatography.

8. Losartan free acid prepared by the process of claim 1 and having less than about 0.05 area-percent of 2-n-Butyl-4-chloro-1[[2′-(1H-tetrazole-5-yl)[1,1′-biphenyl]-4-yl] methyl]-1H-imidazole-5-methoxy methyl ether, as measured by high performance liquid chromatography.

9. Losartan free acid prepared by the process of claim 1 and being substantially free of 2-n-Butyl-4-chloro-1[[2′-(1H-tetrazole-5-yl)[1,1′-biphenyl]4-yl] methyl]-1H-imidazole-5-methoxy methyl ether.

10. The process of claim 1, comprising reacting trityl losartan with aqueous hydrochloric acid.

11. Losartan potassium prepared from the losartan free acid of claim 1.

12. Losartan potassium prepared from the losartan free acid of claim 6.

13. Losartan potassium prepared from the losartan free acid of claim 7.

14. Losartan potassium prepared from the losartan free acid of claim 8.

15. Losartan potassium prepared from the losartan free acid of claim 9.

16. Losartan potassium prepared from losartan free acid being prepared by the process of claim 10.

17. A process for preparing losartan potassium, comprising reacting trityl losartan with an aqueous acid comprising methanesulfonic acid, trifluoroacetic acid, trifluoromethanesulfonic acid, sulfuric acid, phosphoric acid, benzenesulfonic acid, p-toluenesulfonic acid, p-chlorobenzene-sulfonic acid, hydrochloric acid, acetic acid, or formic acid.

18. Losartan potassium containing less than about 1.2 area-percent of 2-n-Butyl-4-chloro-1[[2′-(1H-tetrazole-5-yl)[1,1′-biphenyl]-4-yl] methyl]-1H-imidazole-5-methoxy methyl ether, as determined by high performance liquid chromatography.

19. The losartan potassium of claim 18, containing less than about 0.15 area-percent of 2-n-Butyl-4-chloro-1[[2′-(1H-tetrazole-5-yl)[1,1′-biphenyl]4-yl] methyl]-1H-imidazole-5-methoxy methyl ether, as determined by high performance liquid chromatography.

20. The losartan potassium of claim 18, containing less than about 0.05 area-percent of 2-n-Butyl-4-chloro-1[[2′-(1H-tetrazole-5-yl)[1,1′-biphenyl]-4-yl] methyl]-1H-imidazole-5-methoxy methyl ether, as determined by high performance liquid chromatography.
